(19) United States
(12) Patent Application Publication
(10) Pub. No.: US 2024/0026454 A1
(43) Pub. Date: Jan. 25, 2024

(54) SINGLE NUCLEOTIDE POLYMORPHISM MARKER FOR PREDICTING RISK OF ALZHEIMERS DISEASE AND USE THEREOF

(71) Applicants: SEOUL NATIONAL UNIVERSITY R&DB FOUNDATION, Seoul (KR); INDUSTRY-ACADEMIC COOPERATION FOUNDATION, CHOSUN UNIVERSITY, Gwangju (KR)

(72) Inventors: Sung Ho Won, Seoul (KR); Jun Young Park, Seoul (KR); Kun Ho Lee, Gwangju (KR)

(21) Appl. No.: 18/254,202

(22) PCT Filed: Oct. 18, 2021
(86) PCT No.: PCT/KR2021/014469

§ 371 Date: May 24, 2023

(30) Foreign Application Priority Data
Nov. 25, 2020 (KR) 10-2020-0160077

(51) Int. Cl. C12Q 1/6883

(52) U.S. Cl. CPC C12Q 1/6883; C12Q 2600/118; C12Q 2600/156

(57) ABSTRACT

The present invention relates to: a method for providing information that enables early diagnosis and prediction of a risk group for Alzheimer's disease by providing an SNP marker that can predict high risk of Alzheimer's disease in Korean people; a composition for predicting the risk of Alzheimer's disease; and a microarray and a kit including the composition.

### TECHNICAL FIELD

The present invention relates to a method of predicting the risk of developing Alzheimer's disease by identifying a specific single nucleotide polymorphism (SNP) with a significant correlation with the risk of developing Alzheimer's disease, a composition for predicting the risk of developing Alzheimer's disease, which includes a polynucleotide, a polypeptide, an antibody, or cDNA, which is able to identify the SNP, and a microarray and kit including the same.

### BACKGROUND ART

Alzheimer's disease (AD) is a progressive disorder that causes cognitive impairment and memory loss. Major risk factors for AD include age, family history, and lifestyle. Regarding AD, genome-wide association studies (GWAS) have found more than 30 independent loci, but more than half of the phenotypic variations still remain unexplained. Detection of additional AD risk loci may be enhanced through studies of diverse groups verified by not only GWAS focused on African Americans (Non-Patent Document 1), Hispanics (Non-Patent Documents 2 and 3), Japanese (Non-Patent Documents 4 and 5) and Chinese (Non-Patent Document 6), but also a transethnic approach (Non-Patent Document 7). Studies of diverse populations may utilize population-specific variations and allele frequency differences that often cause variable intensities in association signals. In addition, the effects of disease susceptibility loci may be controlled by environmental risk factors that are differently exposed depending on a population.

Meanwhile, in the case of humans, there is a variation with a frequency of approximately once per 1,000 bases, which is called a single nucleotide polymorphism (SNP), and a 5% polymorphism is referred to as a common polymorphism, and a 1 to 5% polymorphism is referred to as a rare polymorphism. Currently, many experimental techniques have been developed to analyze the entire human base sequence, and among the techniques, GWAS has been used to study many diseases.

GWAS is generally conducted under the assumption that common diseases are associated with common variants, and it is thought that the problem of ‘missing heritability’ arises in such study. The ‘missing heritability’ is a phenomenon that occurs when individual genes cannot explain all phenotypes such as diseases or behaviors, and is an aspect that a disease is determined by a combination of all genotypes. Recently, to compensate for this problem, gene-environment interaction and gene-gene interaction analyses have been widely used.

### RELATED ART DOCUMENTS

### Non-Patent Documents

(Non-Patent Document 1) Reitz, C., Jun, G., Naj, A., Rajbhandary, R., Vardarajan, B. N., Wang, L. S., Valladares, O., Lin, C. F., Larson, E. B., Graff-Radford, N. R., et al. (2013). Variants in the ATP-binding cassette transporter (ABCA7), apolipoprotein E 4, and the risk of late-onset Alzheimer disease in African Americans. Jama 309, 1483-1492.
(Non-Patent Document 2) Lee, J. H., Cheng, R., Barral, S., Reitz, C., Medrano, M., Lantigua, R., Jimenez-Velazquez, I. Z., Rogaeva, E., St George-Hyslop, P. H., and Mayeux, R. (2011). Identification of novel loci for Alzheimer disease and replication of CLU, PICALM, and BIN1 in Caribbean Hispanic individuals. Arch Neurol 68, 320-328.
(Non-Patent Document 3) Vardarajan, B. N., Barral, S., Jaworski, J., Beecham, G. W., Blue, E., Tosto, G., Reyes-Dumeyer, D., Medrano, M., Lantigua, R., Naj, A., et al. (2018). Whole genome sequencing of Caribbean Hispanic families with late-onset Alzheimer's disease. Ann Clin Transl Neurol 5, 406-417.
(Non-Patent Document 4) Miyashita, A., Koike, A., Jun, G., Wang, L. S., Takahashi, S., Matsubara, E., Kawarabayashi, T., Shoji, M., Tomita, N., Arai, H., et al. (2013). SORL1 is genetically associated with late-onset Alzheimer's disease in Japanese, Koreans and Caucasians. PLoS One 8, e58618.
(Non-Patent Document 5) Asanomi, Y., Shigemizu, D., Miyashita, A., Mitsumori, R., Mori, T., Hara, N., Ito, K., Niida, S., Ikeuchi, T., and Ozaki, K. (2019). A rare functional variant of SHARPIN attenuates the inflammatory response and associates with increased risk of late-onset Alzheimer's disease. Mol Med 25, 20.
(Non-Patent Document 6) Zhou, X., Chen, Y., Mok, K. Y., Zhao, Q., Chen, K., Chen, Y., Hardy, J., Li, Y., Fu, A. K. Y., Guo, Q., et al. (2018). Identification of genetic risk factors in the Chinese population implicates a role of immune system in Alzheimer's disease pathogenesis. Proceedings of the National Academy of Sciences 115, 1697.
(Non-Patent Document 7) Jun, G. R., Chung, J., Mez, J., Barber, R., Beecham, G. W., Bennett, D. A., Buxbaum, J. D., Byrd, G. S., Carrasquillo, M. M., Crane, P. K. et al. (2017). Transethnic genome-wide scan identifies novel Alzheimer's disease loci. Alzheimer's Dement 13, 727-738.

### DISCLOSURE

### Technical Problem

The present invention is directed to providing a method of predicting the risk of developing Alzheimer's disease (AD) by identifying a specific single nucleotide polymorphism (SNP) having a significant correlation with the risk of AD in a genetic sample obtained from a patient.

The present invention is also directed to providing a method of providing information for predicting the risk of developing AD by identifying a specific single nucleotide polymorphism (SNP) having a significant correlation with the risk of AD in a genetic sample obtained from a patient.

The present invention is also directed to providing a microarray for predicting the risk of developing AD, including a polynucleotide, a polypeptide, an antibody against the polynucleotide or polypeptide, or cDNA thereof, which is able to identify a specific SNP.

The present invention is also directed to providing a kit for predicting the risk of developing AD, including a polynucleotide, a polypeptide, an antibody against the polynucleotide or polypeptide, or cDNA thereof, which is able to identify a specific SNP.

### Technical Solution

AD is caused by environmental and genetic factors, and preventing AD by predicting genetic factors in advance is effective. In the present invention, a powerful model for predicting AD may be presented using gene-gene interaction.

Accordingly, to find an SNP that appears specifically in the development of AD in Koreans, the present inventors carried out genome-wide association studies (GWAS) by comparing Korean AD patients with mild cognitive impairment (MCI) patients and cognitively normal (CN) subjects and conducted association analysis. As a result, the SNP that specifically appears in Korean patients with AD was identified, and thus the present invention was completed.

Hereinafter, the configuration of the present invention will be described in detail.

The present invention provides a method of predicting the risk of developing AD, which includes confirming whether the base at position 820, represented by NCBI refSNP ID: rs77359862, in SHARPIN is substituted in a genetic sample obtained from a patient.

The genetic sample refers to DNA or RNA that can be isolated from all cells such as blood, skin cells, mucosal cells, and hair of a subject (patient). A method of extracting DNA or RNA from the corresponding cells is not particularly limited, and any technique known in the art or any commercially-available kit for extracting DNA or RNA can be used.

The subject (patient) may include a subject who is determined to have or suspected of having AD. The subject may be a vertebrate, including mammals, amphibian, reptiles, birds, etc., and particularly, a mammal. For example, the subject may be Homo sapiens.

In one embodiment, the subject (patient) may be a Korean. While the corresponding variant is rarely found in the Western population (allele rate: 0.01%), among East Asians, particularly, among Koreans, since approximately 2% of the population have the allele, the subject may be an East Asian, particularly, a Korean. Therefore, this variant may be used as an analytic indicator to predict the risk of developing AD in Koreans.

The “gene” used herein may be used interchangeably with the term “polynucleotide” and “nucleic acid.” The gene includes a DNA fragment involved in the production of a polypeptide chain, and the DNA fragment may include not only regions before and after a coding region, for example, a promoter and the 3′-untranslated region, but also an intervening sequence (intron) between individual coding fragments (exons).

In the present specification, a “gene mutation” is a change in a codon specifying an amino acid due to a variation in a part of the DNA sequence of a wild-type gene, and may include one or more mutations. For example, the gene mutation may include one or more mutations selected from the group consisting of a truncating mutation, a missense mutation, a nonsense mutation, a non-stop mutation, a frame shift mutation, an in-frame mutation, a splice mutation, and a splice region mutation. Preferably, the gene mutation is a missense mutation. The missense mutation is expressed as “(amino acid type) amino acid position (new amino acid type),” for example, R274W may mean that arginine at position 274 of a specific amino acid sequence was replaced with tryptophan.

The “risk of developing” or “possibility of developing” a disease may mean a relative risk of developing AD, and particularly, the likelihood of progressing to AD.

The “prediction” used herein may mean not only determining the possibility of developing AD through the confirmation of the presence or characteristics of a pathological condition, but also drug responsiveness, tolerance, etc. after AD treatment.

The “SHANK associated RH domain interactor (SHARPIN)” used herein refers to a gene encoding a protein of 40 kDa or less, inhibiting β1 and β2 integrin activation in leukocytes by binding to the α1 and α2 integrin tails at a conserved membrane-proximal residue (W/yKXGFFKR). The SHARPIN may be SHARPIN derived from a mammal, and preferably, a human, or from a human-like lineage, and a variant thereof. The human-like lineage refers to other mammals whose gene or mRNA has 80% or more sequence similarity to the human SHARPIN or mRNA derived therefrom, and may specifically include a human, a primate, and a rodent. In one embodiment, a gene encoding SHARPIN may be a sequence disclosed in NCBI Accession No. NM_000008.11, NM_000081.7, NM_005106.4, or NM_041761.1, but the present invention is not limited thereto. In addition, the protein encoded by the gene may be a sequence disclosed in NCBI Accession No. NP_112236.3, NP_079616.2, NP_112415.1 or NP_001267344.1, but the present invention is not limited thereto.

Specifically, the base at position 820, G, on the SHARPIN sequence is substituted with A, which may be represented by NCBI refSNP ID: rs77359862.

The method of providing information according to the present invention may further include predicting that, when the base at position 820 is A, that is, the base at position 820, G, of wild-type SHARPIN is substituted with A, the risk of developing AD is higher, compared to when the base at position 820 is G.

As described above, when the base substitution represented by NCBI refSNP ID: rs77359862 is confirmed, the amino acid at position 274 of SHARPIN changes from arginine (R) to tryptophan (W).

The polynucleotide according to the present invention or a complementary polynucleotide thereof may consist of 10 or more, preferably, 10 to 100, more preferably, 20 to 80, and even more preferably, 40 to 60 consecutive bases, but the present invention is not limited thereto.

The “polynucleotide” used herein generally refers to any polyribonucleotide or polydeoxyribonucleotide, which may be unmodified RNA or DNA, or modified RNA or DNA. Therefore, for example, the polynucleotide as defined in the specification includes, but is not limited to, single- and double-stranded DNA, DNA having single- and double-stranded domains, single- and double-stranded RNA, and RNA having single- and double-stranded domains, a hybrid molecule including DNA and RNA that are single-stranded or more typically double-stranded, or include single- and double-stranded domains. Accordingly, DNA or RNA with a backbone modified for stability or a different reason is a “polynucleotide” which is the same term intended by the specification. In addition, DNA or RNA having an unconventional base such as inosine or a modified base such as a tritiated base may be included in the “polynucleotide” as defined in the specification. Generally, the term “polynucleotide” includes any chemically, enzymatically and/or metabolically modified form(s) of an unmodified polynucleotide. The polynucleotide may be prepared by a variety of methods, including in vitro recombinant DNA-mediated techniques, and DNA expression in cells and organisms.

In the present invention, that the SNP marker can be used to predict the risk of developing AD is based on the high probability that a specific base is present at the SNP site as a result of genetic analysis of a group with AD.

In the present invention, the confirming of whether the base at position 820 is substituted in SHARPIN (represented by NCBI refSNP ID: rs77359862) may be performed by amplifying a polymorphic site corresponding to the sequence represented by rs77359862 or hybridizing with a probe. The amplification of a polymorphic site or hybridizing with a probe may use any method known in the art. For example, the method may be a method of amplifying a target nucleic acid through PCR and purifying the resulting product. In addition, for the method, ligase chain reaction (LCR) (Wu and Wallace, Genomics 4, 560(1989), Landegren et al., Science 241, 1077(1988)), transcription amplification (Kwoh et al., Proc. Natl. Acad. Sci. USA 86, 1173 (1989)), self-maintained sequence duplication (Guatelli et al., Proc. Natl. Acad. Sci. USA 87, 1874 (1990)), or nucleic acid-based sequence amplification (NASBA) may be used.

In addition, the confirmation of the variation may be performed by identifying a genotype of the base at position 820. The confirmation of the variation is performed by sequencing, hybridization by a microarray, allele-specific PCR, dynamic allele-specific hybridization (DASH), PCR elongation assay, SSCP, PCR-RFLP analysis, a TaqMan method, an SNPlex platform (Applied Biosystems), mass spectrometry (e.g., MassARRAY system, Sequenom), mini-sequencing, a Bio-Plex system (BioRad), a CEQ and SNPstream system (Beckman), molecular inversion probe array technology (e.g., Affymetrix GeneChip), or BreadArray technology (e.g., Illumina GoldenGate and Infinium analysis), but the present invention is not limited thereto. By the above methods or other methods available to those of ordinary skill in the art, one or more alleles may be identified from polymorphic markers, including a microsatellite, an SNP or other types of polymorphic markers. Determining the base of such a polymorphic site is preferably performed using an SNP chip.

In addition, in the identifying of a genotype, genetic sequencing may be performed. The sequencing may use any method known in the art. For example, the sequencing may be performed using an automatic sequencer, or may be performed by any one or more methods of known methods such as pyrosequencing, polymerase chain reaction-restriction fragment length polymorphism (PCR-RELP), polymerase chain reaction-single strand conformation polymorphism (PCR-SSCP), polymerase chain reaction-specific sequence oligonucleotide (PCR-SSO), an allele specific oligonucleotide (ASO) hybridization combining PCR-SSO and dot hybridization, TaqMan-PCR, MALDI-TOF/MS, rolling circle amplification (RCA), high resolution melting (HRM), primer elongation, Southern blot hybridization, or dot hybridization.

In the present invention, the method of providing information may be effectively used to predict the risk of developing AD in Koreans.

In addition, the present invention provides an SNP gene set for predicting the risk of developing AD, which includes a polynucleotide consisting of 10 or more consecutive bases, including a mutation of the base at position 820, represented by NCBI refSNP ID: rs77359862, in SHARPIN, or a complementary polynucleotide thereof.

The “polymorphism” used herein refers to the presence of two or more alleles at a single gene locus, and the “polymorphic site” refers to a gene locus at which the allele is present. Among polymorphic sites, those in which a single base differs from person to person are called a “single nucleotide polymorphism (SNP).”.

It is presumed that 99.9% of the genome is identical in individuals, and the remaining 0.1% are involved in individual differences related to the risk of developing a specific disease, hypersensitivity thereof, etc., and an SNP is considered to be directly associated with such risk of disease or hypersensitivity. Since SNPs have a high frequency of appearance, and are almost uniformly distributed throughout the genome, they are considered to be highly reliable genetic polymorphisms for studying the relationship between genes and the risk of disease or hypersensitivity. Accordingly, when changes in such SNPs are effectively analyzed, information related to the association with various types of diseases related to natural genetic modification can be effectively analyzed, so SNPs can greatly contribute to screening of genetic diseases and personalized treatment.

In terms of the location of these SNPs, usually highly conserved sequences are present before and after an SNP. SNPs usually occur when one base at a specific position is replaced with another base, but may also occur by deletion or duplication of a nucleotide. Particularly, when an allele frequency is 5% or less, it is referred to as a rare SNP, and when an allele frequency is 5% or more, it is referred to as a common SNP. Rare SNPs may appear differently by ethnicity or race. Depending on whether such rare SNPs, which are systemically defined populations, are defined for all humans or for a specific population, the scope of rare SNPs may change, and it is obvious that even if a variation appears as a common SNP in one group, it may exhibit the aspect of a rare SNP in another group.

The term “allele” refers to multiple versions of one gene present at the same genetic locus on a homologous chromosome. Alleles are also used to indicate polymorphisms, and for example, in the present invention, an SNP that can consist of only two alleles was used as a marker. Accordingly, the SNP used herein has two types of alleles.

The term “rs_id” used herein refers to rs_ID, which is an independent marker assigned to all SNPs initially registered by NCBI, which began accumulating SNP information in 1998. The rs_id shown in tables below refers to an SNP marker, which is the polymorphic marker of the present invention.

In addition, the present invention provides a composition for predicting the risk of developing AD, including a polynucleotide that specifically hybridizes with the above-described polynucleotide.

In the composition, the polynucleotide that specifically hybridizes with the above-described polynucleotide may be a probe or primer.

The “primer” used herein refers to an oligonucleotide, and acts as the starting point of synthesis under the conditions in which the synthesis of a primer elongation product complementary to a nucleic acid chain (template) is induced, for example, the presence of a nucleotide and a polymerizing agent such as a DNA polymerase, and appropriate temperature and pH conditions. Preferably, the primer is a deoxynucleotide, and single-stranded. The primer used herein may include naturally occurring dNMPs (i.e., dAMP, dGMP, dCMP and dTMP), modified nucleotides, or non-naturally occurring nucleotides. In addition, the primer may also include ribonucleotides.

The term “probe” used herein refers to a naturally-occurring or modified monomer or linear oligomer with linkages, including a deoxyribonucleotide and a ribonucleotide, which can be hybridized to a specific nucleotide sequence. Preferably, the probe is single stranded for maximum efficiency in hybridization. The probe is preferably a deoxyribonucleotide.

As the probe used herein, a sequence perfectly complementary to the sequence having an SNP may be used, but a substantially complementary sequence may be also be used within a range that does not interfere with specific hybridization. Preferably, a probe that is used in the present invention includes a sequence that can be hybridized to a sequence having 10 to 30 consecutive nucleotide residues with the SNP of the present invention. More preferably, the 3′ end or 5′end of the probe may have abase complementary to the SNP base. Generally, since the stability of a duplex formed by hybridization tends to be determined by matching of the terminal ends, in the probe with a base complementary to the SNP base at the 3′ or 5′ end, when the terminal parts are not hybridized, the duplex may disintegrate under a stringent condition.

In addition, the present invention provides a composition for predicting the risk of developing AD, including a polypeptide encoded by the polynucleotide or an antibody specific for the polypeptide.

The term “antibody” used herein, as a term known in the art, refers to a specific protein molecule directed against an antigenic site. For the purpose of the present invention, an antibody refers to an antibody that specifically binds to a polypeptide including the SNP marker of the present invention. Such an antibody may be prepared according to a conventional method using a protein, which is encoded by a marker gene cloned in an expression vector according to a conventional method. Here, the antibody may include a partial peptide that can be made from the protein, and the partial peptide of the present invention may include at least 7 amino acids, preferably, 9 amino acids, and more preferably, 12 or more amino acids. The antibody of the present invention may be, but is not particularly limited to, a polyclonal antibody, a monoclonal antibody, a part thereof if it has antigen binding ability, or any immunoglobulin antibody. Further, the antibody of the present invention may also include a specific antibody such as a humanized antibody. The antibody used to detect a marker for predicting the risk of developing AD of the present invention may include an intact form with two full-length light chains and two full-length heavy chains as well as a functional fragment of the antibody molecule. The functional fragment of the antibody molecule means a fragment that possesses an antigen-binding function, for example, Fab, F(ab′), F(ab′)₂ and Fv.

In addition, the present invention provides a kit for predicting the risk of developing AD, including the polynucleotide, a polynucleotide hybridized therewith, a polypeptide encoded thereby, an antibody specific therefor, or cDNA of the polypeptide.

In the present invention, the kit may be a DNA chip, an RT-PCR kit, or a protein chip kit, but the present invention is not limited thereto.

The kit may predict the risk of developing AD by confirming the amplification of a marker for predicting the risk of developing AD, which is an SNP polymorphic marker, or confirming an expression level of the SNP polymorphic marker at the DNA or mRNA level. For example, in the present invention, the kit for measuring the mRNA expression level of the marker for predicting the risk of developing AD may be a kit including essential factors necessary for RT-PCR. An RT-PCR kit may include, in addition to a primer pair specific for a gene of the marker for predicting the risk of developing AD, a test tube or another proper container, a reaction buffer (with various pHs and magnesium concentrations), deoxynucleotides (dNTPs), enzymes including Taq-polymerase and reverse-transcriptase, a DNase or RNase inhibitor, DEPC-water, and deionized water. In addition, the RT-PCR kit may include a primer pair specific for a gene used as a quantitative control. In addition, the kit according to the present invention is preferably a kit for predicting the risk of developing AD, including essential factors necessary for a DNA chip. A DNA chip kit is a tool that enables massive parallel analysis due to hybridization between a nucleic acid on a DNA chip and a complementary nucleic acid included in a solution treated on a chip surface, manufactured by attaching nucleic acid species on generally a flat solid support plate, and typically, a glass surface which is not larger than a microscopic slide in a gridded array. In addition, the kit according to the present invention may be a protein chip kit. The protein chip kit may measure an expression level of a protein consisting of a mutated amino acid sequence. For immunological detection of an antibody, the protein chip kit may include a substrate, a proper buffer solution, a secondary antibody labeled with a chromogenic enzyme or fluorescent material, and a chromogenic substrate. As a chromogenic enzyme, peroxidase or alkaline phosphatase may be used. In addition, as a fluorescent material, FITC or RITC may be used, and as a chromogenic substrate, 2,2′-azino-bis-(3-ethylbenzothiazolin-6-sulfonic acid) (AVTS), o-phenylenediamine (OPD), or tetramethyl benzidine (TMB) may be used.

In the case of the kit of the present invention manufactured as described above, it is very economical because time and costs are reduced, compared with a general method of detecting the mutation of a gene. Thorough investigation of one gene takes days to months on average using a conventional method of detecting a gene mutation, such as a single strand conformational polymorphism (SSCP), a protein truncation test (PTT), cloning, or direct sequencing. In addition, next generation sequencing (NGS) may also be used to quickly and simply examine gene mutations precisely. When mutations are investigated by a conventional analysis method such as SSCP, cloning, direct base sequencing, or a restriction fragment length polymorphism (RFLP), it takes approximately a month to complete the investigation, whereas the kit of the present invention may be used to obtain a result within approximately 10 to 11 hours when sample DNA is prepared, and since a set of primers capable of detecting mutations are integrated on one chip, not only time but also costs may be reduced compared with a conventional method. Compared to a conventional method, since the kit consumes less than half of the reagent cost per experiment on average, considering the labor costs for researchers, even a greater cost-saving effect can be expected.

In addition, the present invention provides a microarray for predicting the risk of developing AD, including the polynucleotide, a polynucleotide hybridized therewith, a polypeptide encoded thereby, an antibody specific therefor, or cDNA of the polypeptide.

The microarray according to the present invention may include a DNA or RNA polynucleotide. The microarray may consist of conventional microarrays, except that the polynucleotide of the present invention is included in a probe polynucleotide. A method of preparing a microarray by immobilizing a probe polynucleotide on a substrate is well known to the art. The probe polynucleotide refers to a hybridizable polynucleotide, such as an oligonucleotide that can sequence-specifically bind to a complementary strand of a nucleic acid. The probe of the present invention is an allele-specific probe, which has a polymorphic site of nucleic acid fragments derived from two members of the same species, so the probe hybridizes to one DNA fragment derived from one member, or does not hybridize to a fragment derived from the other member. In this case, hybridization conditions should be sufficiently stringent to hybridize to only one of alleles by showing significant differences in hybridization strength between the alleles. This can lead to a good hybridization difference between different alleles. The diagnostic method includes detection methods based on the hybridization of nucleic acids such as Southern blotting, and in a method using a DNA chip, alleles may be provided while binding to a substrate of the DNA chip in advance. The hybridization may be performed under stringent conditions, for example, a salt concentration of 1 M or less and a temperature of 25° C. or more.

Hereinafter, the advantages and features of the present invention and the methods of accomplishing the same will become apparent with reference to the detailed description of exemplary embodiments and the accompanying drawings. However, the present invention is not limited to the exemplary embodiments disclosed below, and may be embodied in many different forms. These exemplary embodiments are merely provided to complete the disclosure of the present invention and fully convey the scope of the present invention to those of ordinary skill in the art, and the present invention should be defined by only the accompanying claims.

### Advantageous Effects

The present invention relates to a single nucleotide polymorphism (SNP) gene set, composition, information providing method, and kit, which can predict the risk of developing Alzheimer's disease (AD) in Koreans. Through genome-wide association study (GWAS), SNP variants associated with the risk of developing AD were identified, and thus the SNP variants can be used in diagnosis and the prediction of developing AD in Koreans.

### DESCRIPTION OF DRAWINGS

FIG. 1 shows genome-wide association study (GWAS) results, in which (a) is a Manhattan plot related to the domain of SHARPIN for the hippocampus, (b) shows the parametric analysis result between SHARPIN and rs77359862 in an Alzheimer's disease (AD) state, (c) shows the effect of rs77359862 on the age of AD onset using the Kaplan-Meier survival curve, and (d) shows the minor allele frequency of rs77359862 in SHARPIN in each population. Accordingly, it can be confirmed that rs77359862 is a gene closely related to East Asians, specifically, Koreans, which is not easily found in Westerners.

FIG. 2 shows the association between the degree of amyloid-beta accumulation, cognitive function and cortical atrophy, and the rs77359862 A allele, in which (a) shows a boxplot of rs77359862 in SHARPIN for standardized uptake value ratio (SUVR) of Aβ-PET data, (b) shows a boxplot of rs77359862 in SHARPIN for Seoul Neuropsychological Screening Battery (SNSB) scores, and (c) shows a cortical thinning map for determining which part of the entire brain domain is significantly affected by the rs77359862 variant.

FIG. 3 illustrates the effect of the SHARPIN (R274W) mutation on the HOIP-SHARPIN complex using molecular dynamics (MD) simulation, in which (a) is a domain map of SHARPIN and HOIP proteins, (b) is the WT crystal structure of HOIPUBA-SHARPINUBL (PDB: 5X0W) indicating the location of the Arg274 residue in wild-type (WT) and manually mutated Trp274 (box), (c) shows the root mean square deviation (RMSD) plot indicating the overall global deviation of the protein complex during 60 ns in WT and the mutant, and (d) shows the RMSF plot indicating the fluctuations of each residue of the protein complex during a simulation period of 60 seconds.

FIG. 4 shows the comparison between WT SHARPIN and SHARPIN (R274W) for interaction with HOIP, in which (a) shows the immunoprecipitation results using 293T cells transiently co-transfected with flag-tagged SHARPIN variants (WT and R247W) and Myc-tagged HOIP, and (b) shows the result of immunoprecipitation performed with an anti-flag antibody using the samples used in (a). It can be confirmed that the mutant does not properly bind with HOIP.

FIG. 5 shows the overall analysis flowchart of a protocol according to the present invention.

FIG. 6 shows the MDS plot with three PC scores ((a) PC1 vs PC2; (b) PC2 vs PC3; and (c) PC1 vs PC3).

FIG. 7 shows the Manhattan plot of GWAS for each MRI trait.

FIG. 8 shows the quantile-quantile (QQ) plot of GWAS.

FIG. 9 shows rare variants and the results of genome-based analysis through differential gene expression (DGE) analysis.

FIG. 10 shows the regional plot for top SNPs for SHARPIN in the results of ADNI (a) and UKB (b), which are Westerner datasets.

FIG. 11 shows interactions on the surfaces of WT (a), in-silico mutant complex (b), and WT complex (c).

FIG. 12 shows interactions on the surfaces of WT (a) and mutant complex (b) after simulation.

FIG. 13 shows the electrostatic potential on a complex surface. A change in charges between (a) WT and (b) a mutant is clearly shown.

FIG. 14 shows the surface model for (a) WT and (b) a mutant, colored according to hydrophobicity.

### MODES OF THE INVENTION

Hereinafter, the present application will be described in detail with reference to examples. The following examples merely illustrate the present application, and the scope of the present application is not limited to the following examples.

### EXAMPLES

### [Example 1] Experimental Methods and Conditions

1. Genome-Wide Association Study (GWAS) Participants

A study sample included 4,563 subjects enrolled in the Gwangju Alzheimer's & Related Dementia (GARD) cohort at Chosun University in Gwangju, and the subjects underwent a neuropsychological assessment using clinical dementia rating (CDR) scores and magnetic resonance imaging (MRI). The clinical diagnosis of Alzheimer's disease (AD) status was conducted in accordance with criteria of the National Institute of Neurological and Communicative Disorders and Stroke-Alzheimer Disease and Research Disorders Association (NINCDS-ADRDA). Cognitively normal (CN) subjects had no evidence of neurological disease or impairment in cognitive function or activities of daily life. Subjects with a history of brain MRIs, a history of head trauma or a history of a psychiatric disorder that can affect mental function were excluded. At baseline, there were 1,614 CN subjects, 1,813 mild cognitive impairment (MCI) subjects and 1,136 AD subjects. A subset of 629 CN subjects and 247 MCI subjects had at least one follow-up examination between 2010 and 2020 (mean follow-up interval: 28.6 months). After follow-up, 53 CN subjects and 21 MCI subjects were reclassified as MCI and AD subjects, respectively, thereby preparing a final sample including 1,561 CN subjects, 1,845 MCI subjects, and 1,157 AD subjects.

The study protocol was approved by the Institutional Review Board of Chosun University Hospital, Korea (CHOSUN 2013-21-018-070). All volunteers or authorized guardians for cognitively-impaired persons gave written consent before participation.

2. Genotyping, Quality Control, Imputation, and Procedures for Principal Component Analysis

5,570 subjects were genotyped using an Affymetrix customized SNP chip, KoreanChip. Genotype data was processed using PLINK and ONETOOL. SNPs were eliminated when a genotype call rate was less than 95%, not in Hardy-Weinberg equilibrium (p<1×10⁻⁵), or there was a significant difference (p<1×10⁻⁵) in call rate between CN, MCI and AD, determined by a Chi-square experiment. After applying these filters, 4,563 subjects and 685,742 SNPs remained.

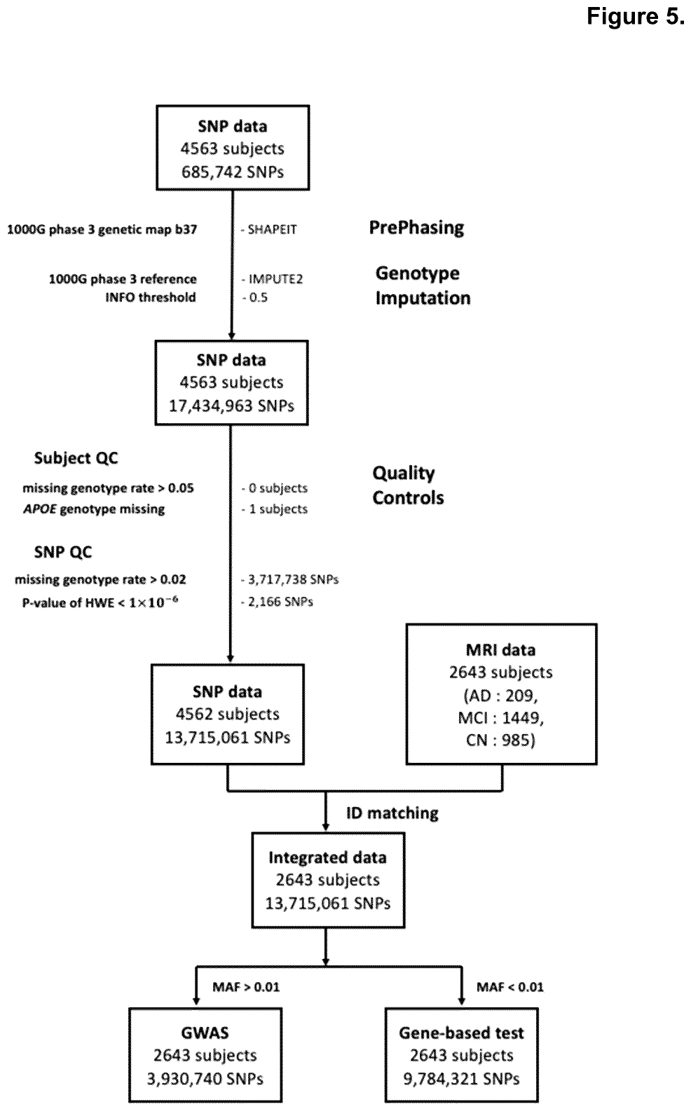

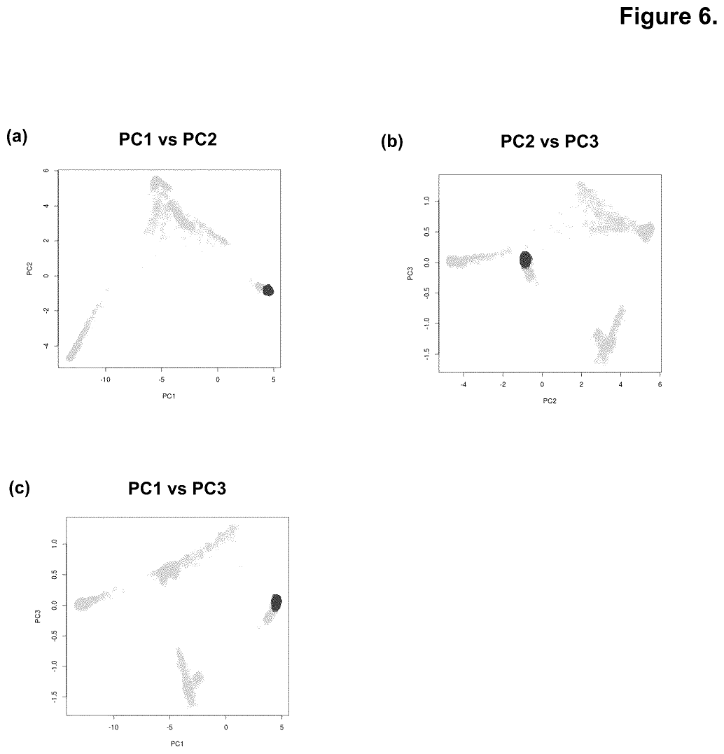

Genotypes were pre-phased using SHAPEIT, and then imputed using a 1000 Genomes Phase 3 reference panel and IMPUTE2. When an INFO score is less than 0.5, a genotype call rate is less than 0.98, or the p-value for HWE is 1×10⁻⁶, the imputed SNPs were eliminated. When the genotype call rate is less than 0.95, or the APOE genotype was missing, the subjects were filtered. As a result, 4,562 subjects and 13,715,061 SNPs remained. The detailed procedure for quality control is illustrated in FIG. 5. Principal component (PC) analysis of ancestors showed that there is almost no evidence of population stratification (FIG. 6).

3. Brain MRI Acquisition and Processing

T1-weighted images (Siemens Healthineers, Erlangen, Germany) were acquired according to the procedures that are described previously, and preprocessed with FreeSurfer V.5.3. The AD-related traits selected for genome-wide association study (GWAS) included measurements of hippocampal volumes and cortical thicknesses of the entorhinal, inferior parietal, middle temporal, and superior frontal regions. MRI traits were available for 209 AD subjects, 1,449 MCI subjects and 985 CN subjects for whom genotype data was available. Compared with 1.5 T (n=688) scanners, since differences between non-normal trait distribution and substantial trait distribution between subjects measured with 3.0 T (n=1,955) were observed, traits for each subgroup were standardized and then transformed by an inverse normal transformation. Descriptive statistics for the MRI traits were obtained using Rex Version 3.0.3 software (RexSoft Inc., Seoul, Korea).

4. Association Analysis Method

GWAS was performed for each trait using PLINK, and linear regression models including imputed SNP genotypes, and covariates for age, sex, and APOE genotype. PC analysis was performed to explain a genetic relationship matrix using EIGENSOFT. The APOE genotype was coded as a class variable with five dummy variables for ε2/ε2, ε2/ε3, ε3/ε3, ε2/ε4, ε3/ε4, and ε4/ε4. The experiment was performed with a total of 3,930,740 SNPs having an allele frequency of less than 0.01. The genome-wide significant threshold was set as p<5.0×10⁻⁸. LocusZoom was used to generate a regional plot, and R software v.3.6 (R Development Core Team, Vienna, Austria) was used to generate QQ, Miami and Manhattan plots. Follow-up analyses were performed on ADNI (n=1,566) and AddNeuroMed (n=288) datasets to replicate or extend genome-wide significant results using similar models like those used in GWAS.

5. Statistical Methods for Testing Association with Measurement of Whole Brain Cortical Thickness

The effect of most SNPs associated with MRI traits was further assessed for their influences on the measurement of whole-brain cortical thicknesses. The SNP genotype effect was analyzed using a dominant model. A general linear model (GLM) was applied to infer pointwise cortical atrophy using a SurfStat toolbox (http://www.math.mcgill.ca/keith/surfstat/) implemented in MATLAB (R2012a, The Mathworks, Natick, MA, USA). Age, sex, APOE ε4 status, and MRI field strength were used as covariates. Random field theory (RFT)-based correction was applied for cortical thickness comparison of various points.

6. Gene-Based Association Analyses Using Rare Variants

Gene-based analyses, including 9,784,321 SNPs with a minor allele frequency (MAF)<0.01, for hippocampal volume and entorhinal thickness were performed using SNP2GENE in Functional Mapping and Annotation of Genome-Wide Association Studies (FUMA), which includes characterization of genomic loci, annotation of candidate SNPs, functional gene mapping, and gene-based analyses. Multiple marker analyses of the genome annotation (MAGMA) tool were used for gene-based analyses. The genome-level significant threshold was set as p<2.6×10⁻⁶, and the covariates were the same as those in GWAS.

7. Mediation Analyses

An SNP showing genome-wide significant association with MRI traits was further evaluated in a sample of 985 CN subjects and 209 AD subjects to determine whether its effect on AD risk is mediated by a specific MRI trait. A mediation model was evaluated using linear regression with AD as an outcome, SNP as a predictor, and an MRI trait variable as a mediator. The model included sex, age, three PCs, and log-transformed intracranial volume (ICV) as covariates. Mediation analyses were conducted using the PROCESS macro implemented in SPSS by selecting four and 10,000 bias-corrected bootstrap samples.

8. Statistical Method for Testing Association Between PET Imaging of AβPET and Cognitive Performance

The accumulation of amyloid beta (Aβ) in the brain was measured on 77 AD subjects, 196 MCI subjects, and 193 CN subjects through positron emission tomography (Aβ-PET). The preprocessing of Aβ-PET images (General Electric Medical Systems, Milwaukee, WI, USA) was performed using a method described previously. The standardized uptake value ratio (SUVR) for Aβ-PET data was defined as the mean activity concentration of six predefined anatomically-related cortical regions of interest (frontal, temporal, parietal, precuneus, anterior cingulate, and posterior cingulate), along with the whole cerebellum used as the reference region. When the SUVR is less than 1.11, it was considered a positive number, and if not, a negative number. The present inventors evaluated the association between the GWS SHARPIN SNP and the derived binary SUVR variables adjusted for age and sex using logistic regression models. The association between the SNP and five domains involved in cognitive ability (attention, frontal/executive function, language, memory, and visuospatial ability) assessed by the Seoul Neuropsychological Screening Battery (SNSB) was tested using linear regression models including covariates of age and sex.

9. Molecular Dynamics Simulation and Analysis

Molecular dynamics (MD) simulation was performed using the crystal structure of the SHARPIN UBL domain binding to the ligand HOIL-1-interacting protein N-terminal UBA domain (HOIP UBA) (PDB ID: 5X0W). Missing residues of the SHARPIN UBL domain (Ala235) and the HOIP UBA domain (gly589=Gly593) in the crystal structure were modeled using the reference SHARPIN sequence and Modweb version r214 in Chimera1.13.1. A selenomethionine residue of the crystal structure was replaced with methionine using CHARMM-GUI. In the mutant variant R274W, to set the Arg274 residue of the SHARPIN UBN domain (from PDB: 5X0W) to Trp using the Pymol v2.3 mutagenesis function, the SHARPIN UBL domain (wt) and the SHARPIN UBL domain (R274W) complexed with the HOIP UBA domain were dissolved with TIP3P water in a PBC rectangular box with minimum 10 Å box-padding and neutralized with 0.15M NaCl. After annealing for 12,000 steps, both the WT and mutant complexes were set to reach a temperature of 310 K for 10,000 steps to minimize energy at 0 K. Subsequently, a 200-ps equilibration step was performed to distribute heat.

10. Immunoprecipitation (IP)

pCMV3flag8SHARPIN (#50014) and HOIP ORF clone (#RC204117) plasmids were purchased from Addgene and Origene, respectively. HOIP was cloned into pcDNA6/myc-His A. The mutant SHARPIN R247W was constructed by site-directed mutagenesis. SHARPIN WT and SHARPIN R247W vectors were transfected into 293T cells with a HOIP-myc vector using TransFectin (#170-3351; Bio-Rad, CA, USA). After 36 hours, the cells were lysed for 1 hour at 4° C. in IP lysis buffer (30 mM Tris-Cl (pH 7.4), 150 mM NaCl, 1% Triton-X100, 1 mM Na3VO4, 50 mM NaF, 1 mM PMSF, 10% glycerol, and 2 mM EDTA). For immunoprecipitation, 1 mg of a cell extract was incubated with 1 μg of anti-c-Myc 9E10 primary antibodies (sc-40; Santa Cruz Biotechnology, Tx, USA) or anti-Flag M2 primary antibodies (F3165; Sigma-Aldrich, MO, USA) overnight at 4° C., and applied to protein A/G agarose beads (P9203; GenDEPOT, TX, USA) for 2 hours. After washing three times, a lysate was subjected to immunoblotting.

11. Immunoblots

The prepared lysate was subjected to sodium dodecyl sulfate polyacrylamide gel electrophoresis, and then transferred to a polyvinylidene difluoride membrane (IPVH00010; Millipore, Billerica, MA). The membrane was blocked with 5% skim milk, washed with 0.1% Tween20 in PBS, and then incubated with anti-c-Myc 9E10 primary antibodies (sc-40; Santa Cruz). Afterward, the membrane was further incubated horseradish peroxidase-conjugated anti-mouse IgG (ab131368; Abcam, Cambridge, UK) at room temperature for 2 hours. Signals were developed using a Clarity Western ECL substrate (1705061; Bio-Rad), and detected by a Fusion Solo S imaging system (VILBER, Collegien, France).

### [Example 2] Results

1. Multiple Genes are Associated with Hippocampal Volume and Entorhinal Thickness in Koreans

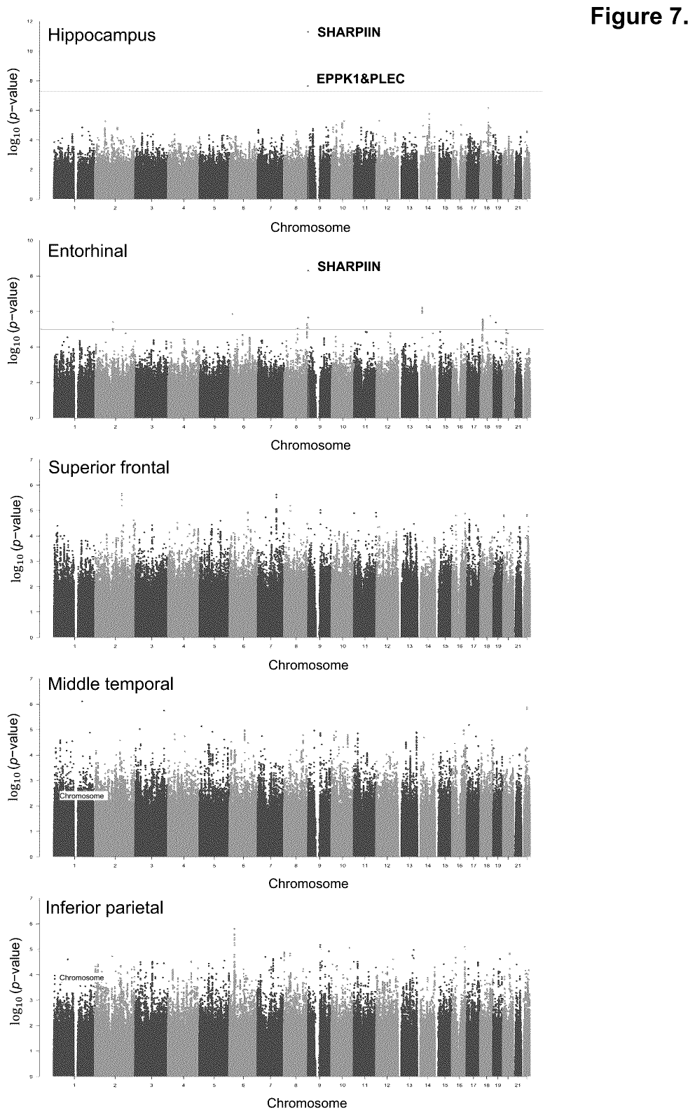

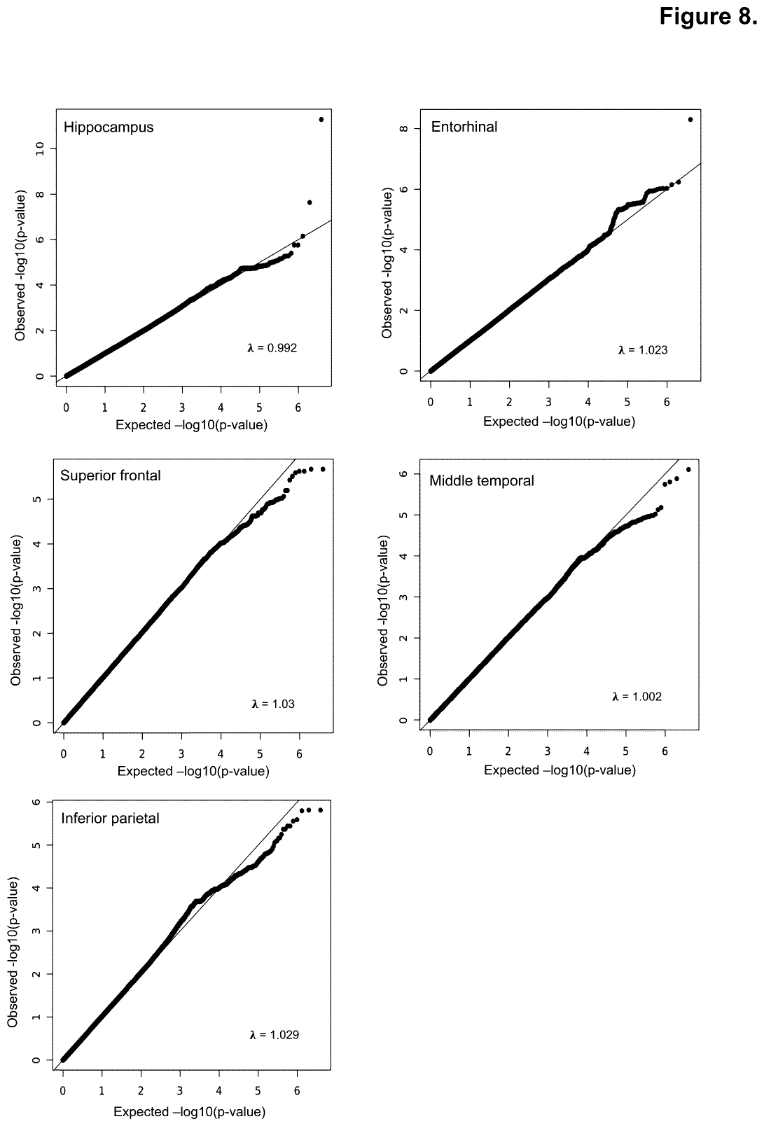

GWAS for five sMRI traits revealed genome-wide significance (GWS, p<5.0×10⁻⁸) and suggestive association (p<1.0×10⁻⁶) for hippocampal volume (HV) and entorhinal thickness (ET) as well as variants in various regions (FIG. 7 and Table 1). There was little evidence of genomic inflation (λ<1.03 for all traits, FIG. 8), and it was shown that both of the analyses were maintained at a nominal significance level. The analysis of association with APOE is summarized in Table 1. The present inventors found that APOE is significant in the entorhinal cortex, inferior parietal, middle temporal and superior frontal, and hippocampal regions, and the results of a likelihood ratio test for ApoE isoform genotypes were p=5.1×10⁻¹¹ and 6.3×10⁻²⁰, respectively. Compared with the genotype ε3ε3, genotypes ε4ε4 and ε3ε4 had significantly different effects on entorhinal cortex, inferior partial, middle temporal and superior frontal, and hippocampal traits, but otherwise had no effect. Non-significance may be partly explained by a small sample size. After adjusting the APOE effect, the results of other SNPs with p<1.0×10⁻⁶ are summarized in Table 1 below.

TABLE 1

| | | Base Pair | | | | | | | | | |
|---|---|---|---|---|---|---|---|---|---|---|---|
| Phenotype | Chromosome | (BP) | SNP | MA | MAF | HWE | I(INFO)/G | β | SE | P-value | Gene |
| Entorhinal | 8 | 145154282 | rs77359862 | A | 0.01 | 0.15 | G | −0.59 | 0.10 | 5.0 × 10⁻⁹ | SHARPIN |
| volume | 14 | 27221601 | rs7160806 | G | 0.39 | 0.97 | I(0.992) | −0.13 | 0.02 | 7.1 × 10⁻⁷ | NOVA1- |
| | | | | | | | | | | | AS1 |
| | 14 | 27219914 | rs1956822 | G | 0.39 | 1 | I(0.995) | −0.13 | 0.02 | 5.8 × 10⁻⁷ | NOVA1- |
| | | | | | | | | | | | AS1 |
| Hippocampal | 8 | 145154282 | rs77359862 | A | 0.01 | 0.15 | G | −0.62 | 0.08 | 5.1 × 10⁻¹² | SHARPIN |
| thickness | 8 | 144984345 | rs80120848 | A | 0.01 | 1 | G | −0.53 | 0.09 | 2.3 × 10⁻⁸ | EPPK1 & |
| | | | | | | | | | | | PLEC |
| | 18 | 48554594 | rs150912768 | T | 0.01 | 1 | I(0.953) | −0.45 | 0.09 | 6.9 × 10⁻⁷ | SMAD4 & |
| | | | | | | | | | | | ELAC1 |

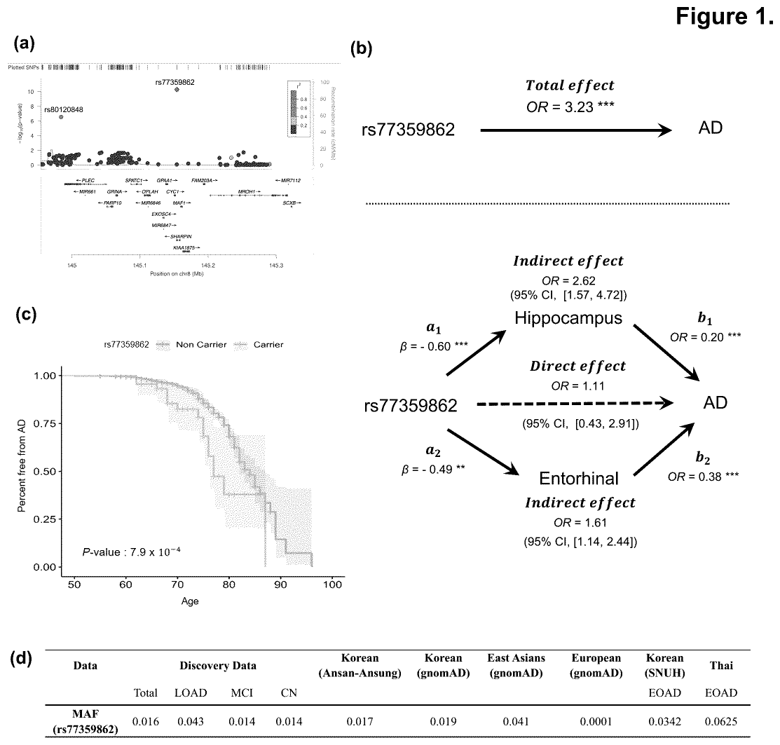

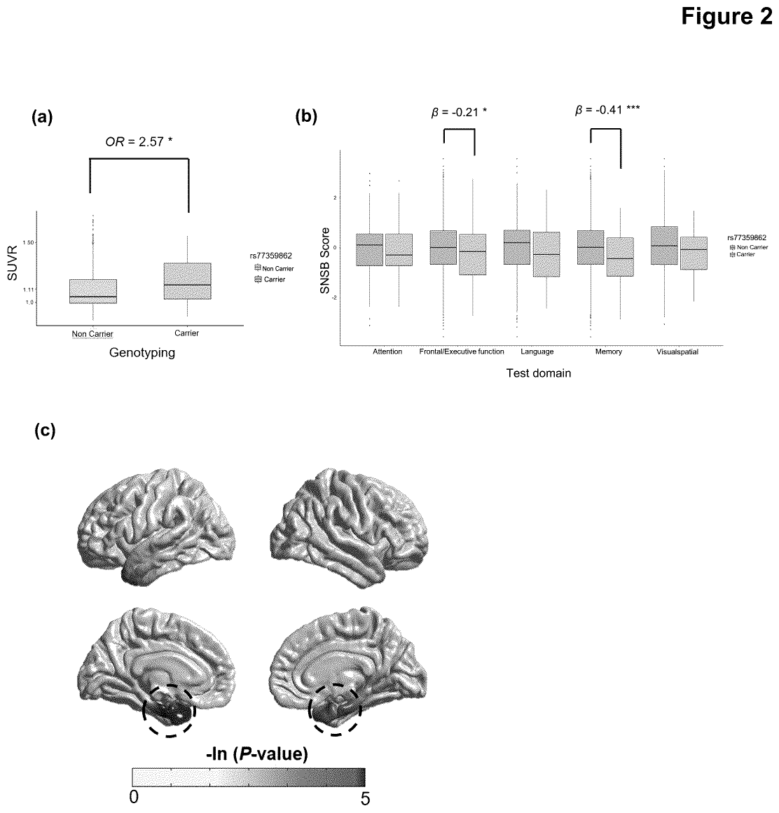

GWS association was also observed in a missense variant (rs77359862) in SHARPIN, together with decreases in ET (p=5.0×10⁻⁹, β=−0.59) and HV (p=5.1×10⁻¹², β=−0.62). Rs80120848 located approximately 5 kb downstream from PLEC was also associated with HV at the GWS level (p=2.3×10⁻⁸, β=−0.53). Since rs80120848 is 189 kb and has some correlation with rs77359862 (r=0.6857), it may not be a dependent association signal (FIG. 1A). For ET with two SNPs (rs7160806, p=7.1×10⁻⁷; rs1956822, p=5.8×10⁻⁷) located in NOVA-AS1 encoding long intergenic non-protein-coding RNA 2588, and HV with rs150912768 (p=6.9×10⁻⁷) located in LOC1053722, which is a gene of unknown function that has an overlapping but reverse-transcribed start site with SMAD4, suggestive associations were observed. The results obtained without non-APOE adjustment are shown in Table 1. Except APOE, NECTIN2 reached suggestive significance threshold for inferior parietal and middle temporal thicknesses, and did not show a change in superior frontal thickness. To determine the effect of rs77359862 on whole-brain atrophy, general linear models (GLMs) for measurement of the whole brain were applied to infer pointwise cortical thicknesses in the whole brain. The present inventors found that 84 carriers of the rs77359862 A allele had significantly greater cortical atrophy (p<0.05) than other cortical atrophy (N=2,559) in the entorhinal cortex and hippocampus (FIG. 2C).

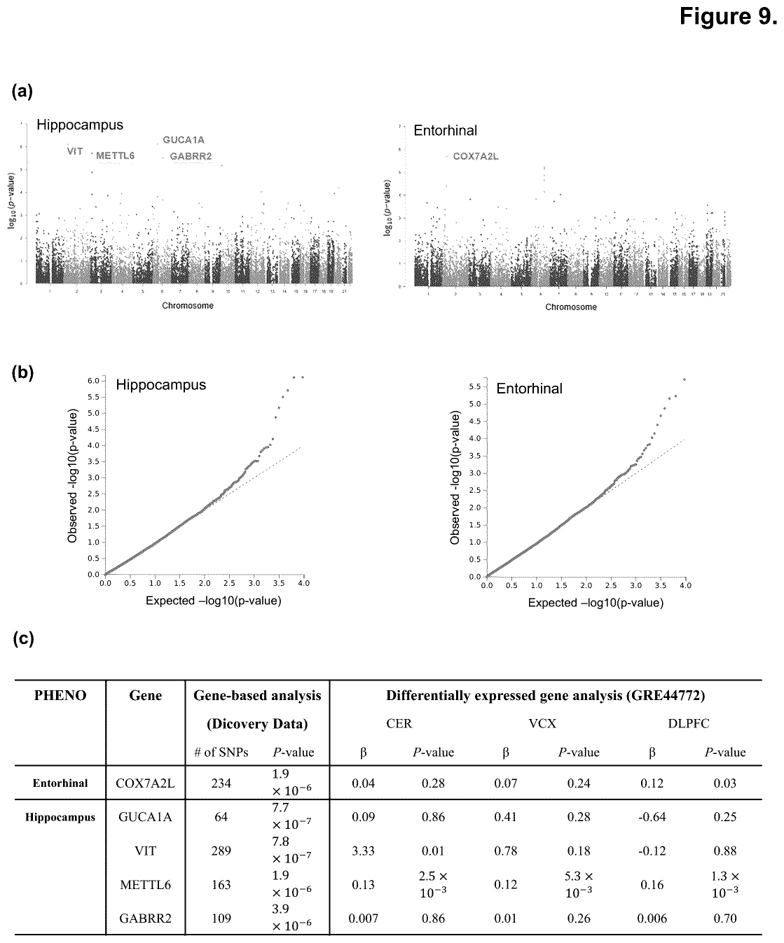

After adjusting the APOE genotype, as a result of gene-based analyses of 18,229 protein-coding genes for rare variants with a minor allele frequency (MAF) of less than 0.01, it was found that there are significant associations (2.7×10⁻⁶) with HV and ET (FIG. 9A) across several genes, and little evidence of genomic inflation including COX7A2L (234 SNPs, p=1.9×10⁻⁶) with ET and genes (GUCA1A (64 SNPs, p=7.7×10⁻⁷), VIT (289 SNPs, p=7.1×10⁻⁹) and METTL6 (163 SNPs, p=1.9×10⁻⁶)) with HV (FIG. 9B). The association between HV and GABRR2 almost reached the gene-wide significance threshold (109 SNPs, p=3.9×10⁻⁶).

2. SHARPIN Missense Variant rs77359862 Indirectly Affects AD Risk by Effect on AD-Related Brain Changes

Next, mediation analyses were performed to estimate an indirect effect of rs77359862 on AD risk through its effect on HV and ET. As shown in FIG. 1B, rs77359862 is significantly associated with AD state (total effect, OR=3.23, and p=3.8×10-4), but the strength of the relationship was weakened (OR=1.11, p=0.82) after adjusting the association with HV and ET. This means that the mechanism based on the effect of rs77359862 on AD risk is mediated by its direct contribution to neurodegeneration particularly in the hippocampal and entorhinal cortex regions. With respect to the total indirect effect of rs77359862 on AD (rs77359862→hippocampus and entorhinal cortex→AD), the indirect effect via the hippocampus (rs77359862→hippocampus→AD) accounted for 67%, and the indirect effect via the entorhinal cortex (rs77359862→entorhinal cortex→AD) accounted for 33%. The estimated odds ratio (OR) value of the total indirect effect of rs77359862 was 4.20 (95% confidence interval (CI): [1.91, 10.05]), and the odds ratio (OR) through the hippocampal and entorhinal regions was 1.61 (CI: [1.14, 2.44]). The above results means that the rs77359862 A allele increases AD risk via the hippocampus by 160% and 61%, respectively.

3. SHARPIN Missense Variant rs77359862 is Associated with AD-Related Clinical Measurement and Biomarker

The present inventors evaluated whether the association of the rs77359862 missense variant is related with several measures of cognitive function using a linear regression model including covariates for age and sex. The association was not observed in measurement of memory (β=−0.41, p=0.0001) and frontal/executive function (β=−0.21, p=0.04), attention (β=−0.09, p=0.38), language (β=−0.18, p=0.09) or visuospatial ability (β=−0.10, p=0.33) (FIG. 2B). Subsequently, the effect of rs77359862 on the age of onset of AD symptoms was investigated using the Kaplan-Meier approach for estimating a survival curve. This analysis showed that AD onset in individuals with the rs77359862 mutant variant was on average 1.5 years earlier than those with a G allele (log=rank test p=7.9×10⁻⁴, FIG. 1C).

The present inventors also investigated the effect of rs77359862 on the progression throughout clinical stages leading to AD in a subset of 876 subjects classified as CN or MCI subjects at baseline, followed by longitudinally tracing for an average of 28.8 months. Within this group, 53 CN and 21 MCI participants (8.4% of total) were converted into MCI and AD participants, respectively (Table S4). The frequency (6/74=8.1%) of the mutant allele among converts was higher than that among non-converts (26/802=3.2%). As a result of analyzing the effect of the rs77359862 genotype on the likelihood of conversion using a proportional hazard model adjusting the APOE genotype, participants with the mutant allele were 2.66 times more likely to progress to the next cognitive stage (p=0.023).

The association of rs773959682 with Aβ accumulation in the brain measured by Aβ-PET was evaluated on 77 AD subjects, 196 MCI subjects and 193 CN subjects in the group subjected to Aβ-PET imaging. Aβ levels were determined by calculating a cortical-to cerebellar standardized uptake value ratio (SUVR). 163 of the 466 subjects had positive SUVRs, and 303 thereof had negative SUVRs. As a result of analyzing the associations using logistic regression models with covariates for age and sex, it was proved that carriers of the rs77359862 missense variant have greater Aβ accumulation than non-carriers (p=0.03, odds ratio=2.57; FIG. 2A).

4. Association of HV with SHARPIN Missense Variant rs77359862 in Other Cohorts

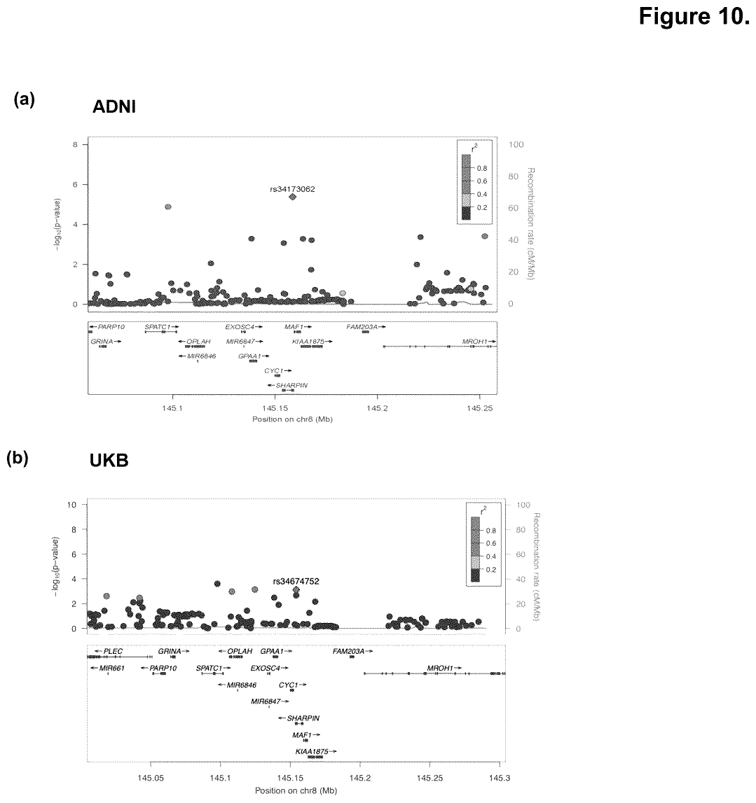

In this study, the frequency of the rs77359862 missense variant was consistently 1% or more in CN Koreans (1.4%), an Ansan-Ansung cohort (1.7%) as well as other East Asians (1.4%) included in the gnomAD database (FIG. 1D). In this study, the present inventors observed a higher frequency of the variant in Koreans with late-onset AD (4.3%) and in 78 early-onset AD (EOAD) patients (3.4%) diagnosed at the Seoul National University Bundang Hospital. This variant was more highly observed in Thailand EOAD patient samples (6.2%). In contrast, the rs77359862 missense variant does not appear in non-Finnish individuals of European ancestry (MAF=0.0001). Accordingly, the rs77359862/AD association may not be evaluated in populations of European ancestry. Therefore, the present inventors hypothesized that other rare functional variants in SHARPIN may be associated with the MRI traits and AD in non-Asians. The present inventors conducted gene-based analyses to confirm the association of HV with SHARPIN including 20 kb beyond the gene boundaries using GWAS for GWS data obtained from the ADNI cohort and NeuroMed cohort data. The gene-based analyses revealed significant association with SHARPIN in both ADNI (86 SNPs; p=0.002) and NeuroMed (93 SNPs; p=0.04), which is more significant in the meta-analysis results of combined samples (FIG. 10A).

5. Change in Stability of SHARPIN Complex Structure Due to rs77359862 Missense Variant

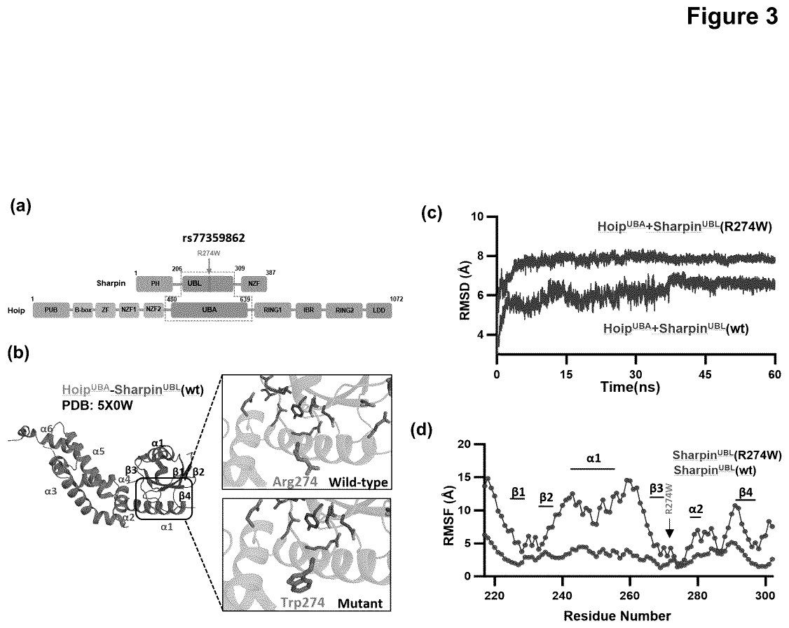

The rs77359862 variant is located in the domain relating to the binding of SHARPIN to the ligand HOIL-1-interacting protein (HOIP), which encodes RING-between-RING (RBR) domain type ε3 ligase. This binding is necessary for SHARPIN-mediated activation of HOIP, which is an important stage for forming a linear ubiquitin assembly complex (LUBAC). Sequence similarity between the reference protein sequence (NP_112236: SHARPIN [Homo sapiens]) and the mapped protein 5X0W_B was identical, accounting for 26% of the reference sequence. The binding site of these two proteins are the HOIP N-terminal UBA domain (HOIP^{UBA}) and the SHARPIN UBL domain (SHARPIN^{UBL}) (FIG. 3A).

In the rs77359862 variant, polar Arg274 is replaced with hydrophobic Trp (NP_112236.3: p.Arg274Trp) and the variant is located in SHARPIN^{UBL} (FIG. 3B). It was considered that this switch in the chemical properties of an amino acid on the surface affects the stability of the bound HOIP^{UBA}-SHARPIN^{UBL} compl3x (FIG. 3B). To understand the effect of this variant, a molecular dynamic (MD) simulation for the WT complex (PDB: 5X0W) and an in-silico SHARPIN mutant (R274W) complex was performed, and the structural change within 60 ns was compared. As shown in FIG. 3C, root mean square deviation (RMSD) analysis revealed that two complexes (HOIP^{UBA}-SHARPIN^{UBL} and HOIP^{UBA}-SHARPIN^{UBL} (R274W)) are stable after the initial 10 ns of the run. The global RMSD value of the mutant HOIP^{UBA}-SHARPIN^{UBL} (R274W) was 2 to 3 Å higher than the WT complex over time. This is caused by the fluctuation of structural factors including α1, α2 and β4 of the mutant SHARPIN^{UBL} (R274W) complex deduced using a root mean square fluctuation (RSMF) plot (FIG. 3D).

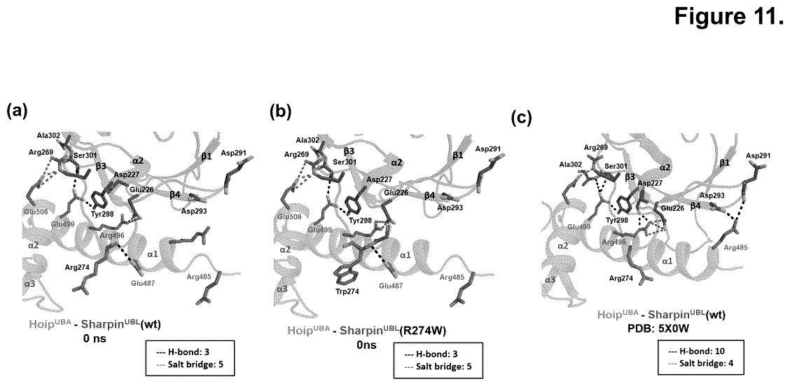

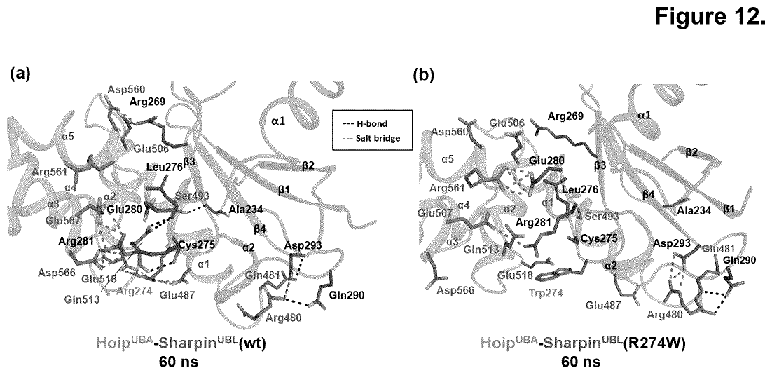

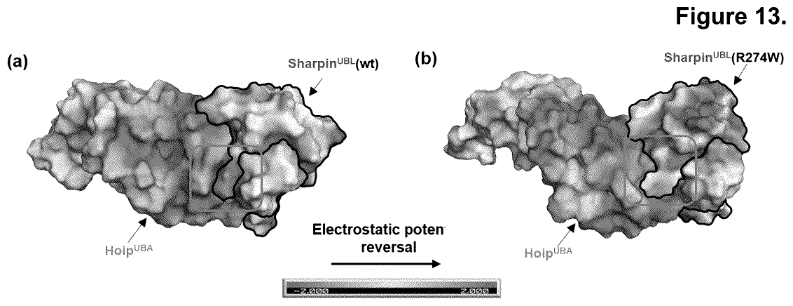

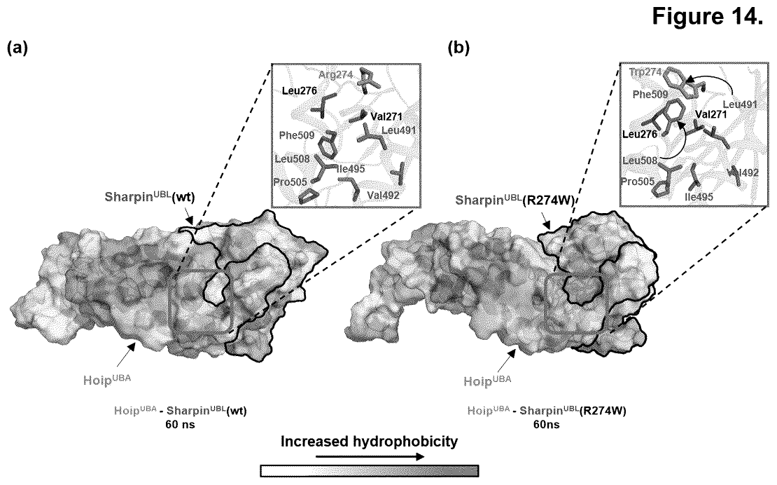

Interactions on the surface between WT HOIP^{UBA} and SHARPIN^{UBL} were strengthened by residues that contribute to hydrogen bonds and salt bridges (FIG. 1D). In 60 ns MD simulation, the WT surface was several times stronger with 6 hydrogen bonds and 19 salt bridges, indicating that the binding energy between the two proteins greatly increased (FIGS. 11B-C, and FIG. 12A). Interestingly, Arg274, which is a residue in the loop (β3-α2) of SHARPIN, formed three salt bridges with Glu518 at α3 of HOIP, increasing the intermolecular interaction at the surface between SHARPIN and HOIP during the simulation (FIG. 12A). The role of Arg27 in stabilization of the complex structure of WT HOIP^{UBA} and SHARPIN^{UBL} was clearly observed through MD simulation (FIG. 12A). Here, the residue underwent a conformational change to link with HOIP Glu518, but did not interact with other amino acids in the crystal structure (FIG. 11C). On the other hand, the SHARPIN^{UBL} (R274W) mutant did not stabilize the surface during simulation. This may be inferred by the apparent decrease in numbers of hydrogen bonds and salt bridges between HOIP^{UBA}-SHARPIN^{UBL} (R274W, two hydrogen bonds and 8 salt bridges) (FIGS. 11B and 12B). In addition, due to the side chain characteristics of the R274W mutant, in which positively charged arginine is replaced with non-polar tryptophan, the electrostatic potential on the surface was reversed along the binding interface of SHARPIN^{UBL} (R274W) (FIGS. 13A and 13B). The charge on the surface became similar in both proteins, so the interaction force may be weakened. Therefore, such observation implied that the reduction in hydrogen bonds and salt bridges having the reversed electrostatic property probably destabilizes and separates the HOIP^{UBA}-SHARPIN^{UBL} (R274W) complex during stimulation, whereas the complex remains even after 60 ns although the interaction seemed weakened. Interestingly, a change in hydrophobic patch between the two proteins was observed in a mutant simulation model (FIGS. 14A and 14B). During simulation, Phe509 of HOIP^{UBA} became closer to the replaced hydrophobic tryptophan in the mutant SHARPIN^{UBL} (R274W), forming π-π stacking with the indole ring of the tryptophan. This allowed the hydrophobic interaction between molecules to increase, and compensated for the loss of another interaction force in the mutant complex.

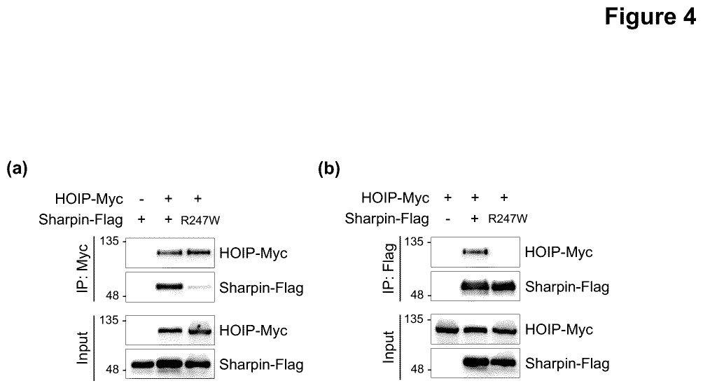

Such observation was further supported by a co-immunoprecipitation (co-IP) experiment. To confirm whether the single point mutation of arginine (R) to tryptophan (W) at position 274 in SHARPIN (R274W) affects interactions with HOIP, flag-tagged SHARPIN WT or R274W mutant protein was co-immunoprecipitated with Myc-tagged wild-type HOIP. The binding between SHARPIN (R274W) and HOIP was decreased by 60% compared with that of WT (FIGS. 4A and 4B). In addition, this study revealed that the SHARPIN mutant R274W may destabilize the interaction between HOIP^{UBA} and SHARPIN^{UBL}, and such destabilization may affect SHARPIN-mediated downstream pathways.

### [Example 3] Conclusion

Previous GWAS has identified many genetic risk loci with GWS for AD, but it has not been consistently replicated. For most GWAS, the case/control study design has limitations such as an experimental group easily contaminated by another neurodegenerative or cerebrovascular disease and a control including future AD cases due to old age. Accordingly, the clinical diagnosis of AD and a quantitative phenotype using brain imaging should be considered in GWAS, and new findings should also be interpreted as brain dysfunction in AD.

In this regard, this study examined GWAS signals for volume and domain changes in five MRI brain domains throughout the clinical spectra of AD, MCI and the control after adjusting the APOE effect. In the study design, the present inventors found that rs77359862 in SHARPIN is a GWS result in the entorhinal cortex and hippocampus (p<5.0×10⁻⁸). The present inventors confirmed that, according to the whole-brain analysis, rs77359862 in SHARPIN is strongly associated with brain atrophy in the entorhinal cortex and HV, showing that a subject with rs77359862 A allele exhibits greater hippocampal atrophy than a subject with a major allele. In addition, the significant association of HV with other SHARPIN variants in ADNI and AddNeuroMed cohorts was found. In addition, according to Soheili-Nezhad et al. [Reference 1], GWAS for the entorhinal cortex was performed using UK Biobank (UKB) [Reference 2] cohort data (N=8,428), it was found that rs34173062 located 4,325 base pairs apart from rs77359862 is significantly associated with the thicknesses of the right and left entorhinal cortices (p=0.002 and 8.6×10⁻⁴, respectively). Even in previous meta-GWAS using Fundacio ACE (GR@ACE), the International Genetics of Alzheimer's project (IGAP), and UKB data, SHARPIN significance was supported (SNP=rs34674752, OR=1.13, p=1.0×10⁻⁹) [Reference 3]. The result suggested that SHARPIN is associated with AD in all three large cohorts recruited in the UK, USA, and Korea.

In addition, the mediation analysis revealed that the SHARPIN variant increases AD risk via the entorhinal cortex and hippocampus (OR=4.2). The PET findings by the present inventors suggested that rs77359862 is critically involved in AD, and functionally affects Aβ accumulation, which is a major component of amyloid plaque on PET images related to the frontal lobe and memory. According to Jung et al. [Reference 4], it can be seen that execution and memory, not language or visuospatial impairment, had a higher risk of cognitive decline, and rs77359862 is significantly associated with executive and memory abilities. Finally, the present inventors prospectively observed that CN or MCI patients with the rs77359862 A allele are much more likely to regress to MCI or AD, respectively. These findings correspond to the pathology of AD, in which neurons and connections in the memory-related brain domains associated with the entorhinal cortex and hippocampus are damaged by amyloid accumulation. In addition, these variants may be associated with EOAD due to duplication with EOAD patients in other datasets from Korea and Thailand, and may play a critical role in Asian populations.

SHARPIN is a component of the linear ubiquitin assembly complex (LUBAC), together with HOIP suppressing NF-kb signaling (PMID: 21811235). To study the mutant (R274W) effect of the SHARPIN UBL domain on complex formation with HOIP, 60 ns MD simulation at 310K was performed using the crystal structure (PDB: 5X0W) for both WT and the mutant variant (R274W). The MD analysis by the present inventors strongly suggested that the mutant complex HOIP UBA domain and the SHARPIN UBL domain (R274W) can destabilize the complex on the surface due to the following reasons. First, the RMSD plot revealed that overall global variance is higher in mutant variants than in WT. Compared to WT, the atomic fluctuation of α1 in the mutant SHARPIN UBL domain (R274W) may explain the higher RMSD. Second, the stable interaction at the interface between the HOIP UBA domain and the SHARPIN UBL domain in the WT complex was disrupted in the mutant. Interactions based on the numbers of hydrogen bonds and salt bridges on the surface are largely broken in the R274W mutant. Third, the electrostatic potential holding both proteins was reversed at the surface by replacing polar arginine with non-polar hydrophobic tryptophan. Therefore, the complex may be dissociated during simulation. However, the two proteins are held together as an unstable complex due to a hydrophobic patch conserved with the additional hydrophobic tryptophan (R274W). In the mutant variant, Trp274 may improve the hydrophobic interaction between the HOIP UBA domain and the SHARPIN UBL domain by the surface hydrophobic patch, π-π interactions, and the interaction of HOIP^{UBA} with adjacent Phe509. Indeed, the physical interaction between HOIP and R274W mutant SHARPIN was greatly reduced compared to the interaction with WT SHARPIN. This unstable complex may affect downstream SHARPIN-mediated NF-kB signaling pathways.

In the nervous system, NF-kB signaling plays a crucial role in the pathophysiology of AD, including neuroinflammation, memory consolidation deficits, Aβ clearance, and neuronal cell death (PMID: 20066105). A rare functional variant of SHARPIN was previously found in Japanese people, and is associated with an increase in late-onset AD risk. The SHARPIN mutant showed a reduction in NF-kB activation in HEK293 cells. In addition, SHARPIN is abundant at synaptic sites of mature neurons, and co-exists with SHANK1 (PMID: 11178875). It is well known that activated NF-kB can move from the activated synapse to the soma, which is essential for long-term memory. The reduction in neuronal NF-kB activity by the SHARPIN variant may inhibit an anti-apoptosis pathway and lead to apoptosis or necrosis in neurons (PMID: 2006615, PMID: 30467385). According to a recent study, the knock-down of SHARPIN using siRNA inhibits Aβ-induced phagocytosis in macrophages, supporting a significant increase in amyloid plaque accumulation in a subject with the R274W mutant SHARPIN.

It seems that rare variants (MAF <0.01) have large and essential effects on identifying that inheritable traits of AD are missing. Through gene-based tests, COX7A2L, GUCA1A, VIT, GABRR2, and METTL6 were found as significant rare variants. AD patients are deficient in cytochrome C oxidase (COX), which is the family gene of COX7A2A, in both peripheral and brain tissue. This may play an important role in bioenergetic deficits in AD. VIT is known to be involved in brain asymmetry. GABRR encodes gamma-aminobutyric acid (GABA) receptor subunit rho-2 and is an important gene in the hippocampus. GABA, which is the major inhibitory neurotransmitter in the brain, is widely distributed in neurons of the cortex and contributes to many cortical functions by binding to a GABA receptor, which is a ligand-gated chloride channel. Accordingly, GABRR2 is involved in general cognitive ability.

### REFERENCES

1: Soheili-Nezhad, S., Jahanshad, N., Guelfi, S., Khosrowabadi, R., Saykin, A. J., Thompson, P. M., Beckmann, C. F., Sprooten, E., and Zarei, M. (2019). A Non-Synonymous SHARPIN Variant is Associated with Limbic Degeneration and Family History of Alzheimer's Disease. bioRxiv, 196410.
2: Sudlow, C., Gallacher, J., Allen, N., Beral, V., Burton, P., Danesh, J., Downey, P., Elliott, P., Green, J., Landray, M., et al. (2015). UK biobank: an open access resource for identifying the causes of a wide range of complex diseases of middle and old age. PLoS Med 12, e1001779-e1001779.
3: de Rojas, I., Moreno-Grau, S., Tesi, N., Grenier-Boley, B., Andrade, V., Jansen, I., Pedersen, N. L., Stringa, N., Zettergren, A., Hernandez, I., et al. (2020). Common variants in Alzheimer's disease: Novel association of six genetic variants with AD and risk stratification by polygenic risk scores. medRxiv, 19012021.
4: Jung, Y. H., Park, S., Jang, H., Cho, S. H., Kim, S. J., Kim, J. P., Kim, S. T., Na, D. L., Seo, S. W., and Kim, H. J. (2020). Frontal-executive dysfunction affects dementia conversion in patients with amnestic mild cognitive impairment. Scientific Reports 10, 772.

1. A method of predicting the risk of developing Alzheimer's disease (AD), comprising:
for a genetic sample obtained from a patient,
confirming whether the base at position 820 is substituted (represented by NCBI refSNP ID: rs77359862) in SHARPIN.

2. The method of claim 1, wherein, when the base at position 820 is A, rather than G, it is predicted that the risk of developing AD is higher.

3. The method of claim 1, wherein, when the substitution of the base represented by NCBI refSNP ID: rs77359862 occurs, the amino acid at position 274 of the SHARPIN protein changes from arginine (R) to tryptophan (W).

4. The method of claim 1, which is for predicting the risk of developing AD in Koreans.

5. A single nucleotide polymorphism (SNP) gene set for predicting the risk of developing Alzheimer's disease (AD), comprising:
a polynucleotide consisting of 10 or more consecutive bases, comprising a mutation of the base at position 820, represented by NCBI refSNP ID: rs77359862, in SHARPIN, or a complementary polynucleotide thereof.

6. The SNP gene set of claim 1, which is for predicting the risk of developing AD in Koreans.

7. A composition for predicting the risk of developing Alzheimer's disease, comprising:
a polynucleotide that specifically hybridizes with a polynucleotide consisting of 10 or more consecutive bases, comprising a mutation of the base at position 820, represented by NCBI refSNP ID: rs77359862, in SHARPIN, or a complementary polynucleotide thereof.

8. The composition of claim 7, wherein the polynucleotide that specifically hybridizes therewith is a probe or primer.

9. A composition for predicting the risk of developing Alzheimer's disease, comprising:
a polypeptide encoded by a polynucleotide consisting of 10 or more consecutive bases, comprising a mutation of the base at position 820, represented by NCBI refSNP ID: rs77359862, in SHARPIN, or a complementary polynucleotide thereof; or an antibody specific therefor.

10. A microarray for predicting the risk of developing Alzheimer's disease, comprising:
a polynucleotide consisting of 10 or more consecutive bases, comprising a mutation of the base at position 820, represented by NCBI refSNP ID: rs77359862, in SHARPIN, or a complementary polynucleotide thereof; a polynucleotide that hybridizes therewith; a polypeptide encoded thereby; an antibody specific therefor; or cDNA of the polypeptide.

11. A kit for predicting the risk of developing Alzheimer's disease, comprising:
a polynucleotide consisting of 10 or more consecutive bases, comprising a mutation of the base at position 820, represented by NCBI refSNP ID: rs77359862, in SHARPIN, or a complementary polynucleotide thereof; a polynucleotide hybridized therewith; a polypeptide encoded thereby; an antibody specific therefor; or cDNA of the polypeptide.
